(19) United States
(12) Patent Application Publication
(10) Pub. No.: US 2025/0095783 A1
(43) Pub. Date: Mar. 20, 2025

(54) METHOD FOR OBTAINING ANTIBODY SEQUENCE

(71) Applicant: BEIJING BAIDU NETCOM SCIENCE TECHNOLOGY CO., LTD., Beijing (CN)

(72) Inventors: Jie GAO, Beijing (CN); Jing HU, Beijing (CN); Xiaomin FANG, Beijing (CN); Xiaonan ZHANG, Beijing (CN)

(73) Assignee: BEIJING BAIDU NETCOM SCIENCE TECHNOLOGY CO., LTD., Beijing (CN)

(21) Appl. No.: 18/967,512

(22) Filed: Dec. 3, 2024

(30) Foreign Application Priority Data
May 28, 2024 (CN) 202410670134.8

(51) Int. Cl. G16B 30/20; G16B 30/10; G16B 40/00

(52) U.S. Cl. CPC G16B 30/20; G16B 30/10; G16B 40/00

(57) ABSTRACT

A method for obtaining an antibody sequence includes: obtaining first features of amino acids at different sequence positions according to an antigen multiple sequence alignment (MSA) sequence, an antibody MSA sequence, and a concatenated sequence of the antigen MSA sequence and the antibody MSA sequence; obtaining second feature of the amino acids at different 3D coordinates according to a graph constructed according to a reference antigen-antibody complex; fusing the first features of amino acids at different sequence positions with the second features of amino acids at 3D coordinates corresponding to the different sequence positions, and obtaining probability information of each of the amino acids at different positions in the antibody sequence according to fused features; and obtaining a target antibody sequence according to the amino acids and their probability information at different positions in the antibody sequence.

### CROSS-REFERENCE TO RELATED APPLICATION

The present disclosure claims the priority and benefit of Chinese Patent Application No. 202410670134.8, filed on May 28, 2024, entitled “METHOD AND APPARATUS FOR OBTAINING ANTIBODY SEQUENCE AND TRAINING ANTIBODY DESIGN MODEL”. The disclosure of the above application is incorporated herein by reference in its entirety.

### TECHNICAL FIELD

The present disclosure relates to the field of artificial intelligence technology, particularly to the field of biological computing technology, and more particularly to a method for obtaining an antibody sequence as well as electronic devices and computer-readable storage media.

### BACKGROUND

Macromolecule drugs possess high targeting and specificity, and the design of macromolecule drugs aims to develop potential therapeutic biomacromolecules such as proteins, antibodies, and nucleic acids. However, current antibody drug design relies on wet lab experiments, which have high development costs and low efficiency.

### SUMMARY

According to an aspect of the present disclosure, a method for obtaining an antibody sequence is provided, which includes: performing Multiple Sequence Alignment (MSA) on an antigen sequence and an initial antibody sequence respectively to obtain an antigen MSA sequence and an antibody MSA sequence; concatenating the antigen MSA sequence and the antibody MSA sequence, and obtaining first features of amino acids at different sequence positions in a concatenated sequence according to respective attribute features of the amino acids in the concatenated sequence, the antigen MSA sequence and the antibody MSA sequence; constructing a graph according to connectivity relationships among amino acids in a reference antigen-antibody complex, and obtaining second features of the amino acids at different 3D coordinates in the reference antigen-antibody complex according to respective attribute features of the amino acids in the graph; fusing the first features of amino acids at different sequence positions with the second features of amino acids at 3D coordinates corresponding to the different sequence positions, and obtaining probability information of each of the amino acids at different positions in the antibody sequence according to the fused features; and obtaining a target antibody sequence according to the amino acids and their probability information at different positions in the antibody sequence.

According to another aspect of the present disclosure, an electronic device is provided, which includes: at least one processor; and a memory communicatively connected to the at least one processor; where the memory stores instructions executable by the at least one processor to perform the above-described method.

According to yet another aspect of the present disclosure, a non-transitory computer-readable storage medium storing computer instructions is provided, where the computer instructions are used to execute the above-described method.

It should be understood that the content described in this section is not intended to identify key or essential features of the embodiments of the present disclosure, nor is it intended to limit the scope of the present disclosure. Other features of the present disclosure will become readily apparent through the following detailed description.

### BRIEF DESCRIPTION OF THE DRAWINGS

The drawings are used to provide a better understanding of the present solution and do not constitute limitations of the present disclosure. Wherein:

FIG. 1 is a schematic diagram according to a first embodiment of the present disclosure;

FIG. 2 is a schematic diagram according to a second embodiment of the present disclosure;

FIG. 3 is a schematic diagram according to a third embodiment of the present disclosure;

FIG. 4 is a schematic diagram according to a fourth embodiment of the present disclosure;

FIG. 5 is a schematic diagram according to a fifth embodiment of the present disclosure; and

FIG. 6 is a block diagram of an electronic device for implementing the method for obtaining an antibody sequence or the method for training an antibody design model according to an embodiment of the present disclosure.

### DETAILED DESCRIPTION OF EMBODIMENTS

The following description of exemplary embodiments of the present disclosure is made with reference to the accompanying drawings, which include various details of the embodiments to aid understanding. These details should be considered merely exemplary. Therefore, those skilled in the art should recognize that various changes and modifications may be made to the embodiments described herein without departing from the scope and spirit of the present disclosure. Similarly, for clarity and conciseness, descriptions of well-known functions and structures are omitted in the following description.

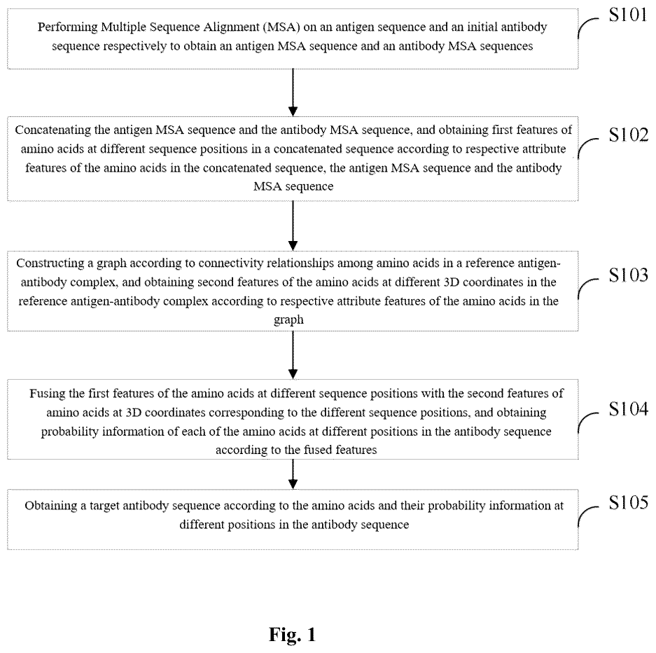

FIG. 1 is a schematic diagram according to a first embodiment of the present disclosure. As shown in FIG. 1, the method for obtaining an antibody sequence in this embodiment specifically includes the following steps:
S101: Performing Multiple Sequence Alignment (MSA) on an antigen sequence and an initial antibody sequence respectively to obtain an antigen MSA sequence and an antibody MSA sequences;
S102: Concatenating the antigen MSA sequence and the antibody MSA sequence, and obtaining first features of amino acids at different sequence positions in a concatenated sequence according to respective attribute features of the amino acids in the concatenated sequence, the antigen MSA sequence and the antibody MSA sequence;
S103: Constructing a graph according to connectivity relationships among amino acids in a reference antigen-antibody complex, and obtaining second features of the amino acids at different 3D coordinates in the reference antigen-antibody complex according to respective attribute features of the amino acids in the graph;
S104: Fusing the first features of the amino acids at different sequence positions with the second features of amino acids at 3D coordinates corresponding to the different sequence positions, and obtaining probability information of each of the amino acids at different positions in the antibody sequence according to the fused features; and
S105: Obtaining a target antibody sequence according to the amino acids and their probability information at different positions in the antibody sequence.

This method for obtaining an antibody sequence combines two types of information: the first features of amino acids obtained from MSA sequences and the second features obtained from the reference antigen-antibody complex, to achieve the purpose of obtaining a target antibody sequence. This approach can improve the accuracy of the obtained target antibody sequence and thereby increase the success rate of obtaining a target antibody sequence.

In this embodiment, both the antigen sequence and initial antibody sequence are amino acid sequences. An amino acid sequence refers to the linear arrangement of amino acids in a protein molecule, which forms the basis for protein structure and function.

The initial antibody sequence in this embodiment may be a wild-type antibody sequence, which refers to an antibody amino acid sequence that comes directly from the natural immune system without artificial modification, or it may be a Heavy Chain (H chain) and/or a Light Chain (L chain) obtained from the wild-type antibody sequence.

When performing MSA (Multiple Sequence Alignment) on the antigen sequence in step S101, this embodiment may obtain, from a preset sequence database, one amino acid sequence having the highest matching degree with the antigen sequence or multiple amino acid sequences having relatively high matching degrees with the antigen sequence, to serve as the antigen MSA sequence(s) corresponding to the antigen sequence.

If the initial antibody sequence is a wild-type antibody sequence, when performing MSA in step S101, this embodiment may obtain, from a preset sequence database, one amino acid sequence having the highest matching degree with the wild-type antibody sequence, or multiple amino acid sequences having relatively high matching degrees with the wild-type antibody sequence, to serve as the antibody MSA sequence(s) corresponding to the initial antibody sequence.

If the initial antibody sequence is an H chain and/or L chain, when performing MSA in step S101, this embodiment may obtain one amino acid sequence having the highest matching degree with the H chain and/or L chain, or multiple amino acid sequences having relatively high matching degrees with the H chain and/or L chain from a preset sequence database to serve as the antibody MSA sequence(s) corresponding to the initial antibody sequence.

When executing S101, this embodiment may use existing MSA algorithms or MSA search tools to perform MSA on an amino acid sequence, thereby obtaining one or multiple MSA sequences corresponding to the amino acid sequence.

After obtaining the antigen MSA sequence and antibody MSA sequence in S101, this embodiment proceeds to S102 to concatenate the antigen MSA sequence and the antibody MSA sequence, and obtains first features of amino acids at different sequence positions in a concatenated sequence according to respective attribute features of the amino acids in the concatenated sequence, the antigen MSA sequence and the antibody MSA sequence. The first features in this embodiment are used to reflect evolutionary information such as whether an amino acid belongs to a conserved region or a variable region in the sequence.

In this embodiment, respective attribute features of the amino acids include type features of the amino acids, index features of the amino acids, position features of the amino acids, and protein chain category features (where protein chain categories include H chain, L chain, and antigen chain) of the amino acids. It should be understood that the attribute features of the amino acids in this embodiment may be fusion features obtained by combining the above features.

When executing S102, this embodiment may append the antigen MSA sequence to the antibody MSA sequence to form the concatenated sequence.

When obtaining the first features of amino acids at different sequence positions in the concatenated sequence according to respective attribute features of the amino acids in the concatenated sequence, the antigen MSA sequence and the antibody MSA sequence in S102, an implementable method is: obtaining an iterative MSA sequence from the antigen MSA sequence and antibody MSA sequence, where this embodiment may randomly select one MSA sequence from multiple MSA sequences as the iterative MSA sequence; obtaining multiple amino acid pair features according to attribute feature pairs of multiple amino acid pairs contained in the iterative MSA sequence, where this embodiment may use the fusion results between attribute feature pairs as amino acid pair features; using multiple amino acid pair features to update the respective attribute features of the amino acids at different sequence positions in the concatenated sequence; after obtaining updated attribute features of the amino acids at different sequence positions in the concatenated sequence, returning to obtain another iterative MSA sequence; repeating this process until all antigen MSA sequences and antibody MSA sequences have been selected, and taking final updated attribute features as the first features of the amino acids.

In other words, this embodiment updates respective attribute features of the amino acids at different sequence positions in the concatenated sequence using amino acid pair features obtained from attribute feature pairs of amino acid pairs contained in MSA sequences, thereby learning evolutionary information of both antigens and antibodies during the update process. The final updated attribute features are used as the first features of amino acids, enabling the first features to reflect evolutionary information of amino acids and thus improving the accuracy of the obtained first features.

For example, if an iterative MSA sequence contains amino acid 1, amino acid 2, and amino acid 3, and if an amino acid pair (amino acid 1, amino acid 2) is obtained from this iterative MSA sequence, the amino acid pair features are obtained according to the attribute feature pair composed of attribute features of amino acid 1 and amino acid 2.

Additionally, when executing S102, besides using amino acid pair features obtained from MSA sequences to update respective attribute features of the amino acids, this embodiment may also use amino acid pair features obtained from the antigen sequence and/or initial antibody sequence to jointly update the respective attribute features of the amino acids.

Furthermore, when executing S102, this embodiment may also include: inputting the antigen MSA sequence and the antibody MSA sequence into a first feature extraction module of an antibody design model, and obtaining the first features of amino acids at different sequence positions in the concatenated sequence according to a result output by the first feature extraction module, where the training process of the antibody design model will be detailed later.

In other words, a pre-trained antibody design model may be used in this embodiment to obtain the first features, which may improve both the efficiency and accuracy of first feature acquisition.

This embodiment may execute S103 either simultaneously with or after completing S102. In S103, a graph is constructed according to connectivity relationships among amino acids in a reference antigen-antibody complex, and second features of the amino acids at different 3D coordinates in the reference antigen-antibody complex are obtained according to respective attribute features of the amino acids in the graph. The second features in this embodiment are used to reflect structural information of amino acids within the complex when an antibody and an antigen bind.

In this embodiment, the reference antigen-antibody complex may be a wild-type antigen-antibody complex, which refers to a complex formed by the natural binding of an unmodified antibody with its corresponding antigen during the natural immune response process.

The graph constructed in S103 according to the reference antigen-antibody complex contains nodes (representing amino acids) and edges among nodes (representing connectivity relationships among amino acids). The nodes themselves carry node features (i.e., attribute features of the amino acids). It should be understood that the 3D coordinates of amino acids in the reference antigen-antibody complex may be determined according to their positions in the graph.

When obtaining the second features of amino acids at different 3D coordinates in the reference antigen-antibody complex in S103 according to respective attribute features of the amino acids in the graph, an implementable method is: determining adjacent amino acids that have connectivity relationships with each amino acid in the graph; using attribute features of the determined adjacent amino acids to update the attribute feature of each amino acid, obtaining updated attribute feature for each amino acid; determining 3D coordinates of each amino acid in the reference antigen-antibody complex according to its position in the graph, and taking the updated attribute features of amino acids at different positions as the second features of amino acids at different 3D coordinates.

In other words, information propagation is performed in this embodiment through nodes and edges in the constructed graph, attribute features of amino acids at different positions are updated, and then 3D coordinates of amino acids in the reference antigen-antibody complex is determined according to the positions of the amino acids in the graph. This process obtains the second features of amino acids at different 3D coordinates in the complex, enabling the second features to reflect structural information of amino acids within the complex after antibody-antigen binding, thereby improving the accuracy of the obtained second features.

Additionally, when executing S103, this embodiment may also include: inputting the reference antigen-antibody complex into a second feature extraction module of the antibody design model, and obtaining the second features of amino acids at different 3D coordinates in the reference antigen-antibody complex according to a result output by the second feature extraction module, where the training process of the antibody design model will be detailed later.

In other words, a pre-trained antibody design model may be used in this embodiment to obtain the second features, which may improve both the efficiency and accuracy of second feature acquisition.

After obtaining the second features of amino acids in S103, this embodiment executes S104 to fuse the first features of amino acids at different sequence positions with the second features of amino acids at 3D coordinates corresponding to the different sequence positions, and obtains probability information of each of the amino acids at different positions in the antibody sequence according to the fused features.

In this embodiment, different sequence positions correspond to different 3D coordinates, and each sequence position can determine a unique 3D coordinate. Therefore, according to the correspondence between sequence positions and 3D coordinates, this embodiment can determine which first and second features should be fused.

For example, if there are sequence positions 1 and 2, and 3D coordinates 1 and 2, where sequence position 1 corresponds to 3D coordinate 1 and sequence position 2 corresponds to 3D coordinate 2, then when executing S104, this embodiment can fuse the first feature of amino acid at sequence position 1 with the second feature of amino acid at 3D coordinate 1, and fuse the first feature of amino acid at sequence position 2 with the second feature of amino acid at 3D coordinate 2.

The probability information obtained in S104 for each of the amino acids at different positions in the antibody sequence reflect the probability of amino acids appearing at different positions in the antibody sequence. The higher the probability value at a current position, the more likely that amino acid is to appear at that position.

Additionally, when executing S104, this embodiment may also include: inputting the first features of amino acids at different sequence positions and the second features of amino acids at 3D coordinates corresponding to the different sequence positions into a feature fusion module of the antibody design model, and obtaining probability information of each of the amino acids at different positions in the antibody sequence according to a result output by the feature fusion module, where the training process of the antibody design model will be detailed later.

In other words, a pre-trained antibody design model may be used in this embodiment to obtain probability information, which may improve both the efficiency and accuracy of probability information acquisition.

After obtaining probability information of each of the amino acids at different positions in the antibody sequence according to the fused features in S104, this embodiment executes S105 to obtain a target antibody sequence according to the amino acids and their probability information at different positions in the antibody sequence.

When executing S105, this embodiment may select an amino acid with the highest probability at each position as the amino acid for that position, thereby completing the selection of amino acids for each position. According to the selection results of amino acids at various positions, a target antibody sequence corresponding to a target antigen is obtained; where the target antigen in this embodiment is an antigen corresponding to the antigen sequence.

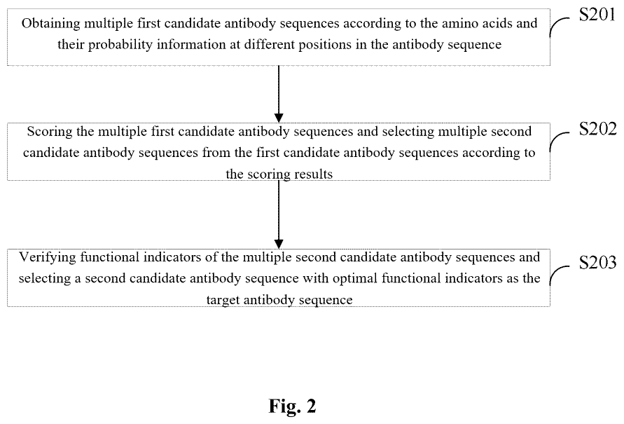

FIG. 2 is a schematic diagram according to a second embodiment of the present disclosure. As shown in FIG. 2, when executing S105 “obtaining a target antibody sequence according to the amino acids and their probability information at different positions in the antibody sequence”, this embodiment specifically includes the following steps:
S201: Obtaining multiple first candidate antibody sequences according to the amino acids and their probability information at different positions in the antibody sequence;
S202: Scoring the multiple first candidate antibody sequences and selecting multiple second candidate antibody sequences from the first candidate antibody sequences according to the scoring results; and
S203: Verifying functional indicators of the multiple second candidate antibody sequences and selecting a second candidate antibody sequence with optimal functional indicators as the target antibody sequence.

In practical scenarios, the target antibody sequence obtained from amino acids corresponding to the highest probability information at different positions may not necessarily have optimal functional indicators. Therefore, this embodiment constructs multiple first candidate antibody sequences according to probability information of amino acids at different positions, then obtains multiple second candidate antibody sequences according to the scoring results of the first candidate antibody sequences, and finally selects the sequence with optimal functional indicators from the second candidate antibody sequences as the target antibody sequence, thereby improving the accuracy of the obtained target antibody sequence.

When executing S201, this embodiment may construct, in descending order of probability, a first candidate antibody sequence with amino acids having the highest probability at different positions, a first candidate antibody sequence with amino acids having the second-highest probability at different positions, and so on. Alternatively, multiple first candidate antibody sequences may be constructed randomly according to the probability information corresponding to amino acids.

When executing S202, preset computational methods such as AlphaFold may be used in this embodiment to score the first candidate antibody sequences, for example, using the pLDDT (Predicted Local Distance Difference Test) value output by AlphaFold as the scoring result for first candidate antibody sequences. When executing S202, the top N-scoring first candidate antibody sequences may be taken as second candidate antibody sequences, where N is a positive integer greater than or equal to 2.

When executing S203, wet lab experiments may be used in this embodiment to verify functional indicators such as neutralizing activity of the second candidate antibody sequences, and then select the second candidate antibody sequence with the highest neutralizing activity as the target antibody sequence.

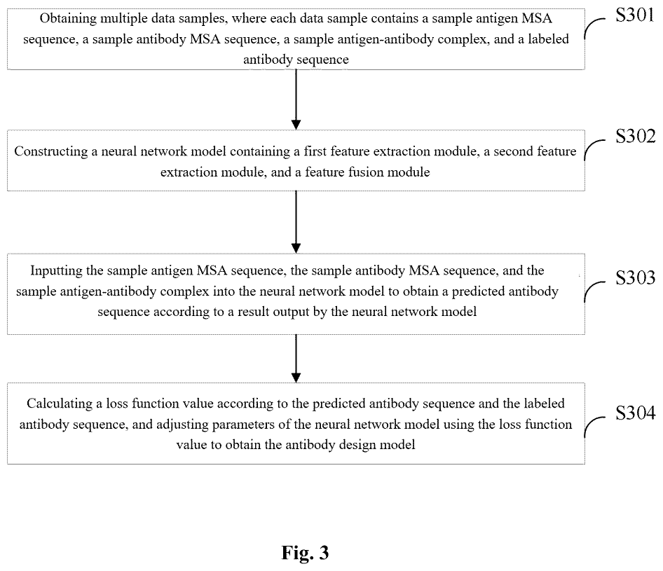

FIG. 3 is a schematic diagram according to a third embodiment of the present disclosure. As shown in FIG. 3, the method for training an antibody design model in this embodiment specifically includes the following steps:
S301: Obtaining multiple data samples, where each data sample contains a sample antigen MSA sequence, a sample antibody MSA sequence, a sample antigen-antibody complex, and a labeled antibody sequence;
S302: Constructing a neural network model containing a first feature extraction module, a second feature extraction module, and a feature fusion module;
S303: Inputting the sample antigen MSA sequence, the sample antibody MSA sequence, and the sample antigen-antibody complex into the neural network model to obtain a predicted antibody sequence according to a result output by the neural network model; and
S304: Calculating a loss function value according to the predicted antibody sequence and the labeled antibody sequence, and adjusting parameters of the neural network model using the loss function value to obtain the antibody design model.

In other words, an antibody design model is trained in this embodiment with multiple acquired data samples. The trained model may extract first and second features according to input information, then obtain probability information of each of the amino acids at different positions in the antibody sequence according to the first and second features extracted, and finally complete the design of a target antibody sequence according to the obtained probability information. This approach simplifies the design process of target antibody sequence and improves design efficiency of the target antibody sequence.

In this embodiment, the first feature extraction module is a Transformer-based neural network structure, the second feature extraction module is a Graph network structure, and the feature fusion module is a Masked Language Model (MLM) composed of multiple Transformer layers.

When executing S303 of inputting the sample antigen MSA sequence, the sample antibody MSA sequence, and the sample antigen-antibody complex into the neural network model, and obtaining the predicted antibody sequence according to the result output by the neural network model, this embodiment may use the following implementation method: inputting the sample antigen MSA sequence and the sample antibody MSA sequence into the first feature extraction module, and obtaining first sample features of amino acids at different sample sequence positions in the sample concatenated sequence output by the first feature extraction module; inputting the sample antigen-antibody complex into the second feature extraction module, and obtaining second sample features of amino acids at different sample 3D coordinates in the sample antigen-antibody complex output by the second feature extraction module; inputting the first sample features of amino acids at different sample sequence positions and the second sample features of amino acids at different sample 3D coordinates into the feature fusion module, and obtaining the predicted antibody sequence according to sample probability information of each of the amino acids at different positions in the sample antibody sequence output by the feature fusion module.

It should be understood that the first feature extraction module performs the processing described in S102 of the previous embodiment, the second feature extraction module performs the processing described in S103, and the feature fusion module performs the processing described in S104, which will not be repeated here.

When executing S303 to obtain a predicted antibody sequence according to the sample probability information output by the feature fusion module, the amino acid with the highest probability at each position may be taken as the amino acid for that position, thereby completing the selection of amino acids for each position and obtaining the predicted antibody sequence according to the selection results.

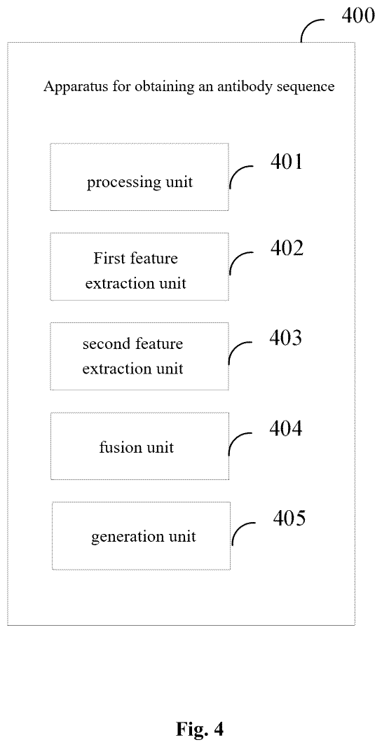

FIG. 4 is a schematic diagram according to a fourth embodiment of the present disclosure. As shown in FIG. 4, the apparatus 400 for obtaining an antibody sequence includes:
A processing unit 401 configured to perform Multiple Sequence Alignment (MSA) on an antigen sequence and an initial antibody sequence respectively to obtain an antigen MSA sequence and an antibody MSA sequence;
A first feature extraction unit 402 configured to concatenate the antigen MSA sequence and antibody MSA sequence, and obtain first features of amino acids at different sequence positions in a concatenated sequence according to respective attribute features of the amino acids in the concatenated sequence, the antigen MSA sequence and the antibody MSA sequence;
A second feature extraction unit 403 configured to construct a graph according to connectivity relationships among amino acids in a reference antigen-antibody complex, and obtain second features of the amino acids at different 3D coordinates in the reference antigen-antibody complex according to respective attribute features of amino acids in the graph;
A fusion unit 404 configured to fuse the first features of amino acids at different sequence positions with the second features of amino acids at 3D coordinates corresponding to the different sequence positions, and obtain probability information of each of the amino acids at different positions in the antibody sequence according to the fused features; and
A generation unit 405 configured to obtain a target antibody sequence according to the amino acids and their probability information at different positions in the antibody sequence.

When performing MSA (Multiple Sequence Alignment) on the antigen sequence, the processing unit 401 may obtain, from a preset sequence database, one amino acid sequence having the highest matching degree with the antigen sequence or multiple amino acid sequences having relatively high matching degrees with the antigen sequence, to serve as the antigen MSA sequence corresponding to the antigen sequence.

If the initial antibody sequence is a wild-type antibody sequence, when performing MSA, the processing unit 401 may obtain, from a preset sequence database, one amino acid sequence having the highest matching degree with the wild-type antibody sequence or multiple amino acid sequences having relatively high matching degrees with the wild-type antibody sequence, to serve as the antibody MSA sequence corresponding to the initial antibody sequence.

If the initial antibody sequence is an H chain and/or L chain, when performing MSA, the processing unit 401 may obtain, from a preset sequence database, one acid sequence having the highest matching degree with the H chain and/or L chain, or multiple amino acid sequences having relatively high matching degrees with the H chain and/or L chain, to serve as the antibody MSA sequence corresponding to the initial antibody sequence.

The processing unit 401 may use existing MSA algorithms or MSA search tools to perform MSA on amino acid sequences to obtain one or multiple MSA sequences corresponding to the amino acid sequence.

After the processing unit 401 obtains the antigen MSA sequence and antibody MSA sequence, the first feature extraction unit 402 concatenates the antigen MSA sequence and antibody MSA sequence and obtains first features of amino acids at different sequence positions in a concatenated sequence according to respective attribute features of the amino acids in the concatenated sequence, the antigen MSA sequence and the antibody MSA sequence. The first features in this embodiment are used to reflect evolutionary information such as whether an amino acid belongs to a conserved region or a variable region in the sequence.

In this embodiment, respective attribute features of the amino acids include type features of the amino acids, index features of the amino acids, position features of the amino acids, and protein chain category features (where protein chain categories include H chain, L chain, and antigen chain) of the amino acids. It should be understood that the attribute features of the amino acids in this embodiment may be fusion features obtained by combining the above features.

The first feature extraction unit 402 may append the antigen MSA sequence to the antibody MSA sequence to form the concatenated sequence.

When obtaining first features of amino acids at different sequence positions in the concatenated sequence according to respective attribute features of the amino acids in the concatenated sequence, the antigen MSA sequence and the antibody MSA sequence, the first feature extraction unit 402 may use the following implementation method: obtaining an iterative MSA sequence from the antigen MSA sequence and antibody MSA sequence; obtaining multiple amino acid pair features according to attribute feature pairs of multiple amino acid pairs contained in the iterative MSA sequence; using multiple amino acid pair features to update the respective attribute features of the amino acids at different sequence positions in the concatenated sequence; after obtaining updated attribute features of the amino acid at different sequence positions in the concatenated sequence, returning to obtain another iterative MSA sequence; repeating this process until all antigen and antibody MSA sequences have been selected, and taking final updated attribute features as the first features of amino acids.

In other words, the first feature extraction unit 402 updates respective attribute features of the amino acids at different sequence positions in the concatenated sequence using amino acid pair features obtained from attribute feature pairs of amino acid pairs contained in MSA sequences, thereby learning evolutionary information of both antigens and antibodies during the update process. The final updated attribute features are used as the first features of amino acids, enabling the first features to reflect evolutionary information of amino acids and improving the accuracy of the obtained first features.

Additionally, besides using amino acid pair features obtained from MSA sequences to update respective attribute features of the amino acids, the first feature extraction unit 402 may also use amino acid pair features obtained from the antigen sequence and/or initial antibody sequence for joint updating the respective attribute features of the amino acids.

Furthermore, the first feature extraction unit 402 may also include: inputting the antigen MSA sequence and antibody MSA sequence into a first feature extraction module of an antibody design model and obtaining the first features of amino acids at different sequence positions in the concatenated sequence according to a result output by the first feature extraction module, where the training process of the antibody design model will be detailed later.

In other words, the first feature extraction unit 402 may use a pre-trained antibody design model to obtain the first features, which may improve both the efficiency and accuracy of first feature acquisition.

In this embodiment, the second feature extraction unit 403 constructs a graph according to connectivity relationships among amino acids in a reference antigen-antibody complex and obtains second features of the amino acids at different 3D coordinates in the reference antigen-antibody complex according to respective attribute features of the amino acids in the graph. The second features are used to reflect structural information of amino acids within the complex when antibodies and antigens bind.

In this embodiment, the reference antigen-antibody complex may be a wild-type antigen-antibody complex, which refers to a complex formed by the natural binding of an unmodified antibody with its corresponding antigen during the natural immune response process.

The graph constructed by the second feature extraction unit 403 contains nodes (representing amino acids) and edges among nodes (representing connectivity relationships among amino acids). The nodes themselves carry node features (i.e., attribute features of the amino acids). It should be understood that the 3D coordinates of amino acids in the reference antigen-antibody complex may be determined according to their positions in the graph.

When obtaining second features of amino acids at different 3D coordinates in the reference antigen-antibody complex according to respective attribute features of the amino acids in the graph, the second feature extraction unit 403 may use the following implementation method: determining adjacent amino acids that have connectivity relationships with each amino acid in the graph; using attribute features of the determined adjacent amino acids to update the attribute feature of each amino acid, obtaining updated attribute feature of each amino acid; determining 3D coordinates of each amino acid in the reference antigen-antibody complex according to its position in the graph, and taking the updated attribute features of amino acids at different positions as the second features of amino acids at different 3D coordinates.

In other words, the second feature extraction unit 403 performs information propagation through nodes and edges in the constructed graph, updates attribute features of amino acids at different positions, and then determines 3D coordinates of amino acids in the reference antigen-antibody complex according to the positions of the amino acids in the graph. This process obtains the second features of amino acids at different 3D coordinate in the complex, enabling the second features to reflect structural information of amino acids within the complex after antibody-antigen binding, thereby improving the accuracy of the obtained second features.

Additionally, the second feature extraction unit 403 may also include: inputting the reference antigen-antibody complex into a second feature extraction module of the antibody design model and obtaining the second features of amino acids at different 3D coordinates in the reference antigen-antibody complex according to a result output by the second feature extraction module, where the training process of the antibody design model will be detailed later.

In other words, the second feature extraction unit 403 may also use a pre-trained antibody design model to obtain the second features, which improves both efficiency and accuracy of the second feature acquisition.

After obtaining second features of amino acids by the second feature extraction unit 403, the fusion unit 404 fuses the first features of amino acids at different sequence positions with the second features at 3D coordinates corresponding to the different sequence positions, and obtains probability information of each of the amino acids at different positions in the antibody sequence according to the fused features.

In this embodiment, different sequence positions correspond to different 3D coordinates, and each sequence position can determine a unique 3D coordinate. Therefore, according to the correspondence between sequence positions and 3D coordinates, the embodiment can determine which first and second features should be fused.

The probability information obtained by the fusion unit 404 for each of the amino acids at different positions in the antibody sequence reflect the probability of amino acids appearing at different positions in the antibody sequence. The higher the probability value at a current position, the more likely that amino acid is to appear at that position.

Additionally, the fusion unit 404 may also input the first features of amino acids at different sequence positions and the second features of amino acids at 3D coordinates corresponding to the different sequence positions into a feature fusion module of the antibody design model, and obtain probability information of each of amino acids at different positions in the antibody sequence according to a result output by the feature fusion module, where the training process of the antibody design model will be detailed later.

In other words, the fusion unit 404 may use a pre-trained antibody design model to obtain probability information, which improves both efficiency and accuracy of probability information acquisition.

After the fusion unit 404 obtains probability information of each of the amino acids at different positions in the antibody sequence according to the fused features, the generation unit 405 obtains a target antibody sequence according to the amino acids and their probability information at different positions in the antibody sequence.

The generation unit 405 may select an amino acid with the highest probability at each position as the amino acid for that position, thereby completing the selection of amino acids for each position. According to the selection results of amino acids at various positions, a target antibody sequence corresponding to a target antigen is obtained, where the target antigen in this embodiment is an antigen corresponding to the antigen sequence.

When obtaining the target antibody sequence, the generation unit 405 may obtain multiple first candidate antibody sequences according to the amino acids and their probability information at different positions in the antibody sequence; score the multiple first candidate antibody sequences and select multiple second candidate antibody sequences according to the scoring results; and verify functional indicators of the multiple second candidate antibody sequences and select a second candidate antibody sequence with optimal functional indicators as the target antibody sequence.

In practical scenarios, the target antibody sequence obtained from amino acids with highest probability information at different positions may not necessarily have optimal functional indicators. Therefore, the generation unit 405 constructs multiple first candidate sequences according to probability information of amino acids at different positions, obtains multiple second candidate sequences according to the scoring results of the first candidate antibody sequence, and finally selects the sequence with optimal functional indicators from the second candidate antibody sequences as the target antibody sequence, thereby improving accuracy of the obtained target antibody sequence.

The generation unit 405 may construct, in descending order of probability, a first candidate sequence with amino acids having highest probability at different positions, a first candidate sequence with amino acids having the second highest probability at different positions, and so on. Alternatively, multiple first candidate antibody sequences may be constructed randomly according to probability information corresponding to amino acids.

The generation unit 405 may use preset computational methods such as AlphaFold to score the first candidate antibody sequences, for example using pLDDT (Predicted Local Distance Difference Test) values output by AlphaFold as scoring results of the first candidate antibody sequences. The top N-scoring sequences may be taken as second candidate sequences, where N is a positive integer greater than or equal to 2.

The generation unit 405 may use wet lab experiments to verify functional indicators such as neutralizing activity of the second candidate sequences, and then select the second candidate antibody sequence with the highest neutralizing activity as the target antibody sequence.

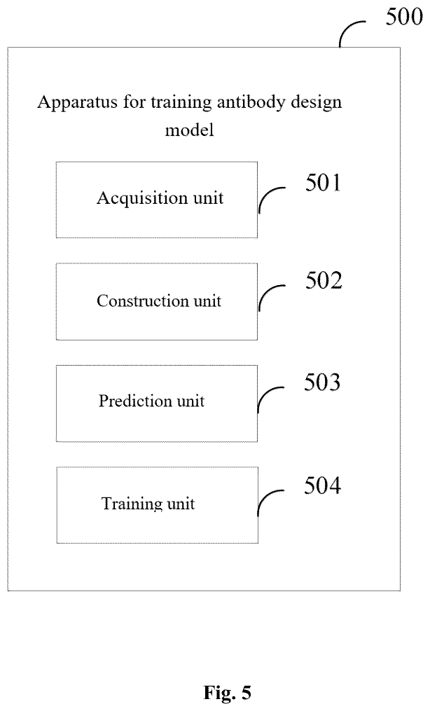

FIG. 5 is a schematic diagram according to a fifth embodiment of the present disclosure. As shown in FIG. 5, the apparatus 500 for training an antibody design model includes:

An acquisition unit 501 configured to obtain multiple data samples, where each data sample contains a sample antigen MSA sequence, a sample antibody MSA sequence, a sample antigen-antibody complex, and a labeled antibody sequence;
A construction unit 502 configured to construct a neural network model containing a first feature extraction module, a second feature extraction module, and a feature fusion module;
A prediction unit 503 configured to input the sample antigen MSA sequence, the sample antibody MSA sequence, and the sample antigen-antibody complex into the neural network model to obtain a predicted antibody sequence according to a result output by the neural network model;
A training unit 504 configured to calculate a loss function value according to the predicted antibody sequence and the labeled antibody sequence, and adjust parameters of the neural network model using the loss function value to obtain the antibody design model.

In other words, this embodiment trains an antibody design model using multiple acquired data samples. The trained model may extract first and second features according to input information, obtain probability information of each of the amino acids at different positions in the antibody sequence according to the first and second features extracted, and finally complete the design of a target antibody sequence according to the probability information obtained. This approach simplifies the design process of the target antibody sequence and improves design efficiency of the target antibody sequence.

In this embodiment, the first feature extraction module is a Transformer-based neural network structure, the second feature extraction module is a Graph network structure, and the feature fusion module is a Masked Language Model (MLM) composed of multiple Transformer layers.

When inputting the sample antigen MSA sequence, the sample antibody MSA sequence, and the sample antigen-antibody complex into the neural network model, and obtaining the predicted antibody sequence according to the result output by the neural network model, the prediction unit 503 may use the following implementation method: inputting the sample antigen MSA sequence and the sample antibody MSA sequence into the first feature extraction module, and obtaining first sample features of amino acids at different sample sequence positions in the sample concatenated sequence output by the first feature extraction module; inputting the sample antigen-antibody complex into the second feature extraction module, and obtaining second sample features of amino acids at different sample 3D coordinates in the sample antigen-antibody complex output by the second feature extraction module; inputting the first sample features of amino acids at different sample sequence positions and the second sample features of amino acids at different sample 3D coordinates into the feature fusion module, and obtaining the predicted antibody sequence according to sample probability information of each of the amino acids at different positions in the sample antibody sequence output by the feature fusion module.

When obtaining the predicted antibody sequence according to sample probability information of each of the amino acids at different positions in the sample antibody sequence output by the feature fusion module, the prediction unit 503 may select the amino acid with the highest probability at each position to complete the selection of amino acids for each position and obtain the predicted antibody sequence according to the selection results of each position.

In the technical solutions of this disclosure, the acquisition, storage, and application of user personal information comply with relevant laws and regulations and do not violate public order and good morals.

According to some embodiments of the present disclosure, an electronic device, a computer-readable storage medium, and a computer program product are also provided.

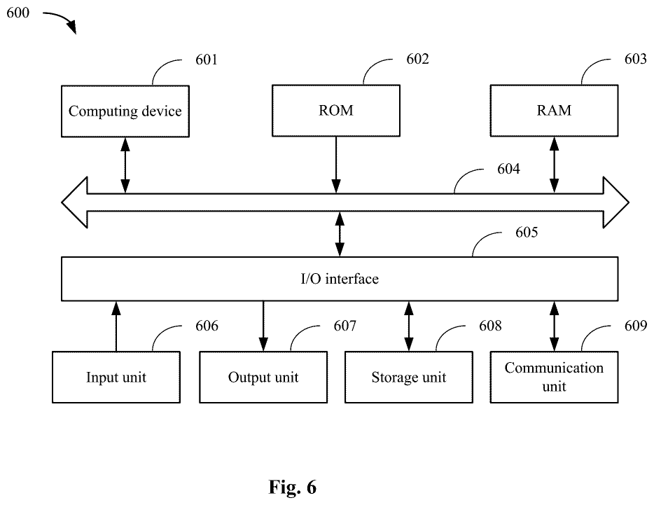

As shown in FIG. 6, a block diagram of an electronic device for implementing the method for obtaining an antibody sequence or the method for training an antibody design model is provided. The electronic device is intended to represent various forms of digital computers, such as laptops, desktop computers, workstations, personal digital assistants, servers, blade servers, mainframes, and other suitable computers. The electronic device may also represent various forms of mobile devices, such as personal digital processors, cellular phones, smartphones, wearable devices, and other similar computing devices. The components shown, their connections and relationships, and their functions are meant as examples only and are not intended to limit implementations of the disclosure described and/or claimed herein.

As shown in FIG. 6, device 600 includes a computing unit 601 that may execute various appropriate actions and processes according to computer programs stored in Read-Only Memory (ROM) 602 or loaded from storage unit 608 to Random Access Memory (RAM) 603. RAM 603 may also store various programs and data needed for device 600 operation. Computing unit 601, ROM 602, and RAM 603 are connected via bus 604. An Input/Output (I/O) interface 605 is also connected to bus 604.

Multiple components in device 600 are connected to I/O interface 605, including: input unit 606, such as keyboard, mouse, etc.; output unit 607, such as various types of displays, speakers, etc.; storage unit 608, such as magnetic disks, optical disks, etc.; and communication unit 609, such as network cards, modems, wireless communication transceivers, etc. Communication unit 609 allows device 600 to exchange information/data with other devices through computer networks like the Internet and/or various telecommunication networks.

Computing unit 601 may be various general-purpose and/or specialized processing components with processing and computing capabilities. Examples of computing unit 601 include but are not limited to Central Processing Units (CPU), Graphics Processing Units (GPU), various specialized Artificial Intelligence (AI) computing chips, computing units running machine learning model algorithms, Digital Signal Processors (DSP), and any appropriate processors, controllers, microcontrollers, etc. Computing unit 601 executes the various methods and processes described above, such as methods for obtaining antibody sequences or training antibody design models. For example, in some embodiments, these methods may be implemented as computer software programs tangibly embodied in machine-readable media, such as storage unit 608.

In some embodiments, parts or all of the computer programs may be loaded and/or installed onto device 600 via ROM 602 and/or communication unit 609. When the computer programs are loaded into RAM 603 and executed by computing unit 601, they may perform one or more steps of the methods described above. Alternatively, in other embodiments, computing unit 601 may be configured to perform these methods through other appropriate means (e.g., through firmware).

The various implementations of the systems and techniques described here may be realized in digital electronic circuitry, integrated circuitry, Field Programmable Gate Arrays (FPGAs), Application Specific Integrated Circuits (ASICs), Application Specific Standard Products (ASSPs), System on Chip (SoC), Complex Programmable Logic Devices (CPLDs), computer hardware, firmware, software, and/or combinations thereof. These various implementations may include implementation in one or more computer programs that are executable and/or interpretable on a programmable system including at least one programmable processor, which may be special or general purpose, capable of receiving data and instructions from, and transmitting data and instructions to, a storage system, at least one input device, and at least one output device.

Program code for implementing the methods of the present disclosure may be written in any combination of one or more programming languages. These program codes may be provided to general-purpose computers, special-purpose computers, or other programmable devices for obtaining antibody sequences or training antibody design models, such that when executed by processors or controllers, they implement the functions/operations specified in the flowcharts and/or block diagrams. The program code may be executed entirely on the machine, partly on the machine, partly on the machine as a stand-alone software package and partly on a remote machine, or entirely on the remote machine or server.

In the context of this disclosure, a machine-readable medium may be a tangible medium that contains or stores programs for use by or in connection with an instruction execution system, apparatus, or device. The machine-readable medium may be a machine-readable signal medium or a machine-readable storage medium. The machine-readable medium may include, but is not limited to, electronic, magnetic, optical, electromagnetic, infrared, or semiconductor systems, apparatus, or device, or any suitable combination thereof. More specific examples of machine-readable storage media would include electrical connections based on one or more wires, portable computer disks, hard disks, Random Access Memory (RAM), Read-Only Memory (ROM), Erasable Programmable Read-Only Memory (EPROM or flash memory), optical fibers, portable Compact Disc Read-Only Memory (CD-ROM), optical storage devices, magnetic storage devices, or any suitable combination thereof.

To provide interaction with users, the systems and techniques described herein may be implemented on a computer having a display device (e.g., CRT (Cathode Ray Tube) or LCD (Liquid Crystal Display) monitor) for displaying information to users, and a keyboard and pointing device (e.g., mouse or trackball) through which users may provide input. Other types of devices may also be used for providing interaction with users; for example, feedback provided to users may be any form of sensory feedback (e.g., visual, auditory, or tactile feedback); and input from users may be received in any form, including acoustic, speech, or tactile input.

The systems and techniques described herein may be implemented in computing systems that include back-end components (e.g., as data servers), middleware components (e.g., application servers), or front-end components (e.g., user computers with graphical user interfaces or web browsers through which users may interact with implementations of the systems and techniques described here), or any combination of such back-end, middleware, or front-end components. The components of the system may be interconnected by any form or medium of digital data communication (e.g., a communication network). Examples of communication networks include Local Area Networks (LAN), Wide Area Networks (WAN), and the Internet.

The computer system may include clients and servers. Clients and servers are generally remote from each other and typically interact through a communication network. The client-server relationship is established by computer programs running on respective computers and having a client-server relationship. The server may be a cloud server, also known as a cloud computing server or cloud host, which is a hosting product in cloud computing service systems that addresses the limitations of traditional physical hosts and VPS (Virtual Private Server) services, such as management difficulties and weak business scalability. The server may also be a distributed system server or a server integrated with blockchain.

It should be understood that the various forms of processes shown above may be reordered, added to, or have steps removed. For example, the steps described in this disclosure may be executed in parallel, sequentially, or in different orders, as long as they achieve the desired results of the technical solutions disclosed herein.

As used in the description herein and throughout the claims that follow, “a”, “an”, and “the” includes plural references unless the context clearly dictates otherwise.

The specific embodiments described above do not constitute limitations on the scope of protection of this disclosure. Those skilled in the art should understand that various modifications, combinations, sub-combinations, and substitutions may be made according to design requirements and other factors. Any modifications, equivalent substitutions, and improvements made within the spirit and principles of this disclosure should be included within its scope of protection.

What is claimed is:

1. A method for obtaining an antibody sequence, comprising:
performing Multiple Sequence Alignment (MSA) on an antigen sequence and an initial antibody sequence respectively to obtain an antigen MSA sequence and an antibody MSA sequence;
concatenating the antigen MSA sequence and the antibody MSA sequence, and obtaining first features of amino acids at different sequence positions in a concatenated sequence according to respective attribute features of the amino acids in the concatenated sequence, the antigen MSA sequence and the antibody MSA sequence;
constructing a graph according to connectivity relationships among amino acids in a reference antigen-antibody complex, and obtaining second features of the amino acids at different 3D coordinates in the reference antigen-antibody complex according to respective attribute features of the amino acids in the graph;
fusing the first features of amino acids at different sequence positions with the second features of amino acids at 3D coordinates corresponding to the different sequence positions, and obtaining probability information of each of the amino acids at different positions in the antibody sequence according to fused features; and
obtaining a target antibody sequence according to the amino acids and their probability information at different positions in the antibody sequence.

2. The method according to claim 1, wherein obtaining the first features of amino acids at different sequence positions in the concatenated sequence according to respective attribute features of the amino acids in the concatenated sequence, the antigen MSA sequence and the antibody MSA sequence comprises:
obtaining an iterative MSA sequence from the antigen MSA sequence and the antibody MSA sequence;
obtaining multiple amino acid pair features according to attribute feature pairs of multiple amino acid pairs contained in the iterative MSA sequence;
updating the respective attribute features of the amino acids at different sequence positions in the concatenated sequence using the multiple amino acid pair features;
returning to the step of obtaining an iterative MSA sequence from the antigen MSA sequence and the antibody MSA sequence after obtaining updated attribute features of the amino acids at different sequence positions in the concatenated sequence; and
repeating the above processes until all antigen MSA sequence and antibody MSA sequence have been selected, and taking final updated attribute features as the first features of the amino acids.

3. The method according to claim 1, wherein obtaining the second features of the amino acids at different 3D coordinates in the reference antigen-antibody complex according to respective attribute features of the amino acids in the graph comprises:
determining adjacent amino acids having connectivity relationships with each amino acid in the graph;
updating attribute feature of each amino acid using attribute features of the adjacent amino acids to obtain updated attribute feature of each amino acid;
determining a 3D coordinate of each amino acid in the reference antigen-antibody complex according to its position in the graph, and taking the updated attribute features of amino acids at different positions as the second features of amino acids at different 3D coordinates.

4. The method according to claim 1, wherein obtaining the target antibody sequence according to the amino acids and their probability information at different positions in the antibody sequence comprises:
obtaining multiple first candidate antibody sequences according to the amino acids and their probability information at different positions in the antibody sequence;
scoring the multiple first candidate antibody sequences and selecting multiple second candidate antibody sequences from the multiple first candidate antibody sequences according to the scoring results; and
verifying functional indicators of the multiple second candidate antibody sequences, and selecting a second candidate antibody sequence with optimal functional indicators as the target antibody sequence.

5. The method according to claim 1, wherein obtaining first features of amino acids at different sequence positions in the concatenated sequence according to respective attribute features of the amino acid in the concatenated sequence, the antigen MSA sequence and the antibody MSA sequence comprises:
inputting the antigen MSA sequence and the antibody MSA sequence into a first feature extraction module of an antibody design model;
obtaining the first features of amino acids at different sequence positions in the concatenated sequence according to a result output by the first feature extraction module.

6. The method according to claim 1, wherein constructing the graph according to connectivity relationships among amino acids in the reference antigen-antibody complex, and obtaining the second features of the amino acids at different 3D coordinates in the reference antigen-antibody complex according to respective attribute features of the amino acids in the graph comprises:
inputting the reference antigen-antibody complex into a second feature extraction module of an antibody design model; and
obtaining the second features of amino acids at different 3D coordinates in the reference antigen-antibody complex according to a result output by the second feature extraction module.

7. The method according to claim 1, wherein fusing the first features of amino acids at different sequence positions with the second features of amino acids at 3D coordinates corresponding to the different sequence positions, and obtaining probability information of each of the amino acids at different positions in the antibody sequence according to fused features comprises:
inputting the first features of amino acids at different sequence positions and the second features of amino acids at 3D coordinates corresponding to the different sequence positions into a feature fusion module of an antibody design model; and
obtaining probability information of each of the amino acids at different positions in the antibody sequence according to a result output by the feature fusion module.

8. The method according to claim 1, wherein the first features are obtained by inputting the antigen MSA sequence and the antibody MSA sequence into a first feature extraction module of an antibody design model;
the second features are obtained by inputting the reference antigen-antibody complex into a second feature extraction module of the antibody design model; and
the probability information of each of the amino acids at different positions are obtained by inputting the first features and the second features into a feature fusion module of the antibody design model.

9. The method according to claim 8, wherein the antibody design model is trained by:
obtaining multiple data samples, wherein each data sample contains a sample antigen MSA sequence, a sample antibody MSA sequence, a sample antigen-antibody complex, and a labeled antibody sequence;
constructing a neural network model containing the first feature extraction module, the second feature extraction module, and the feature fusion module;
inputting the sample antigen MSA sequence, the sample antibody MSA sequence, and the sample antigen-antibody complex into the neural network model, and obtaining a predicted antibody sequence according to a result output by the neural network model; and
calculating a loss function value according to the predicted antibody sequence and the labeled antibody sequence, and adjusting parameters of the neural network model using the loss function value to obtain the antibody design model.

10. The method according to claim 9, wherein inputting the sample antigen MSA sequence, sample antibody MSA sequence, and sample antigen-antibody complex into the neural network model, and obtaining the predicted antibody sequence according to the result output by the neural network model comprises:
inputting the sample antigen MSA sequence and sample antibody MSA sequence into the first feature extraction module, and obtaining first sample features of amino acids at different sample sequence positions in the sample concatenated sequence output by the first feature extraction module;
inputting the sample antigen-antibody complex into the second feature extraction module, and obtaining second sample features of amino acids at different sample 3D coordinates in the sample antigen-antibody complex output by the second feature extraction module; and
inputting the first sample features of amino acids at different sample sequence positions and the second sample features of amino acids at different sample 3D coordinates into the feature fusion module, and obtaining the predicted antibody sequence according to sample probability information of each of the amino acids at different positions in the sample antibody sequence output by the feature fusion module.

11. An electronic device, comprising:
at least one processor; and
a memory communicatively connected to the at least one processor;
wherein the memory stores instructions executable by the at least one processor to perform a method for obtaining an antibody sequence, comprising:
performing Multiple Sequence Alignment (MSA) on an antigen sequence and an initial antibody sequence respectively to obtain an antigen MSA sequence and an antibody MSA sequence;
concatenating the antigen MSA sequence and the antibody MSA sequence, and obtaining first features of amino acids at different sequence positions in a concatenated sequence according to respective attribute features of the amino acids in the concatenated sequence, the antigen MSA sequence and the antibody MSA sequence;
constructing a graph according to connectivity relationships among amino acids in a reference antigen-antibody complex, and obtaining second features of the amino acids at different 3D coordinates in the reference antigen-antibody complex according to respective attribute features of the amino acids in the graph;
fusing the first features of amino acids at different sequence positions with the second features of amino acids at 3D coordinates corresponding to the different sequence positions, and obtaining probability information of each of the amino acids at different positions in the antibody sequence according to fused features; and
obtaining a target antibody sequence according to the amino acids and their probability information at different positions in the antibody sequence.

12. The electronic device according to claim 11, wherein obtaining the first features of amino acids at different sequence positions in the concatenated sequence according to respective attribute features of the amino acids in the concatenated sequence, the antigen MSA sequence and the antibody MSA sequence comprises:
obtaining an iterative MSA sequence from the antigen MSA sequence and the antibody MSA sequence;
obtaining multiple amino acid pair features according to attribute feature pairs of multiple amino acid pairs contained in the iterative MSA sequence;
updating the respective attribute features of the amino acids at different sequence positions in the concatenated sequence using the multiple amino acid pair features;
returning to the step of obtaining an iterative MSA sequence from the antigen MSA sequence and the antibody MSA sequence after obtaining updated attribute features of the amino acids at different sequence positions in the concatenated sequence; and
repeating the above processes until all antigen MSA sequence and antibody MSA sequence have been selected, and taking final updated attribute features as the first features of the amino acids.

13. The electronic device according to claim 11, wherein obtaining the second features of the amino acids at different 3D coordinates in the reference antigen-antibody complex according to respective attribute features of the amino acids in the graph comprises:
determining adjacent amino acids having connectivity relationships with each amino acid in the graph;
updating attribute feature of each amino acid using attribute features of the adjacent amino acids to obtain updated attribute feature of each amino acid;
determining a 3D coordinate of each amino acid in the reference antigen-antibody complex according to its position in the graph, and taking the updated attribute features of amino acids at different positions as the second features of amino acids at different 3D coordinates.

14. The electronic device according to claim 11, wherein obtaining the target antibody sequence according to the amino acids and their probability information at different positions in the antibody sequence comprises:
obtaining multiple first candidate antibody sequences according to the amino acids and their probability information at different positions in the antibody sequence;
scoring the multiple first candidate antibody sequences and selecting multiple second candidate antibody sequences from the multiple first candidate antibody sequences according to the scoring results; and
verifying functional indicators of the multiple second candidate antibody sequences, and selecting a second candidate antibody sequence with optimal functional indicators as the target antibody sequence.

15. The electronic device according to claim 11, wherein obtaining first features of amino acids at different sequence positions in the concatenated sequence according to respective attribute features of the amino acid in the concatenated sequence, the antigen MSA sequence and the antibody MSA sequence comprises:
inputting the antigen MSA sequence and the antibody MSA sequence into a first feature extraction module of an antibody design model;
obtaining the first features of amino acids at different sequence positions in the concatenated sequence according to a result output by the first feature extraction module.

16. The electronic device according to claim 11, wherein constructing the graph according to connectivity relationships among amino acids in the reference antigen-antibody complex, and obtaining the second features of the amino acids at different 3D coordinates in the reference antigen-antibody complex according to respective attribute features of the amino acids in the graph comprises:
inputting the reference antigen-antibody complex into a second feature extraction module of an antibody design model; and
obtaining the second features of amino acids at different 3D coordinates in the reference antigen-antibody complex according to a result output by the second feature extraction module.

17. The electronic device according to claim 11, wherein fusing the first features of amino acids at different sequence positions with the second features of amino acids at 3D coordinates corresponding to the different sequence positions, and obtaining probability information of each of the amino acids at different positions in the antibody sequence according to fused features comprises:
inputting the first features of amino acids at different sequence positions and the second features of amino acids at 3D coordinates corresponding to the different sequence positions into a feature fusion module of an antibody design model; and
obtaining probability information of each of the amino acids at different positions in the antibody sequence according to a result output by the feature fusion module.

18. The electronic device according to claim 11, wherein the first features are obtained by inputting the antigen MSA sequence and the antibody MSA sequence into a first feature extraction module of an antibody design model;
the second features are obtained by inputting the reference antigen-antibody complex into a second feature extraction module of the antibody design model; and
the probability information of each of the amino acids at different positions are obtained by inputting the first features and the second features into a feature fusion module of the antibody design model, and
wherein the antibody design model is trained by:
obtaining multiple data samples, wherein each data sample contains a sample antigen MSA sequence, a sample antibody MSA sequence, a sample antigen-antibody complex, and a labeled antibody sequence;
constructing a neural network model containing the first feature extraction module, the second feature extraction module, and the feature fusion module;
inputting the sample antigen MSA sequence, the sample antibody MSA sequence, and the sample antigen-antibody complex into the neural network model, and obtaining a predicted antibody sequence according to a result output by the neural network model; and
calculating a loss function value according to the predicted antibody sequence and the labeled antibody sequence, and adjusting parameters of the neural network model using the loss function value to obtain the antibody design model.

19. The electronic device according to claim 18, wherein inputting the sample antigen MSA sequence, sample antibody MSA sequence, and sample antigen-antibody complex into the neural network model, and obtaining the predicted antibody sequence according to the result output by the neural network model comprises:
inputting the sample antigen MSA sequence and sample antibody MSA sequence into the first feature extraction module, and obtaining first sample features of amino acids at different sample sequence positions in the sample concatenated sequence output by the first feature extraction module;
inputting the sample antigen-antibody complex into the second feature extraction module, and obtaining second sample features of amino acids at different sample 3D coordinates in the sample antigen-antibody complex output by the second feature extraction module; and
inputting the first sample features of amino acids at different sample sequence positions and the second sample features of amino acids at different sample 3D coordinates into the feature fusion module, and obtaining the predicted antibody sequence according to sample probability information of each of the amino acids at different positions in the sample antibody sequence output by the feature fusion module.

20. A non-transitory computer-readable storage medium storing computer instructions for executing a method for obtaining an antibody sequence, comprising:
performing Multiple Sequence Alignment (MSA) on an antigen sequence and an initial antibody sequence respectively to obtain an antigen MSA sequence and an antibody MSA sequence;
concatenating the antigen MSA sequence and the antibody MSA sequence, and obtaining first features of amino acids at different sequence positions in a concatenated sequence according to respective attribute features of the amino acids in the concatenated sequence, the antigen MSA sequence and the antibody MSA sequence;
constructing a graph according to connectivity relationships among amino acids in a reference antigen-antibody complex, and obtaining second features of the amino acids at different 3D coordinates in the reference antigen-antibody complex according to respective attribute features of the amino acids in the graph;
fusing the first features of amino acids at different sequence positions with the second features of amino acids at 3D coordinates corresponding to the different sequence positions, and obtaining probability information of each of the amino acids at different positions in the antibody sequence according to fused features; and
obtaining a target antibody sequence according to the amino acids and their probability information at different positions in the antibody sequence.
